(19) United States
(12) Patent Application Publication
(10) Pub. No.: US 2025/0103600 A1
(43) Pub. Date: Mar. 27, 2025

(54) MULTI-OMICS BASED TECHNIQUES FOR PRODUCT TARGET DISCOVERY

(71) Applicant: CENTRE DE RECHERCHE ET DE DEVELOPPEMENT DES ANATIDES DU COURTALET, TOULOUSE CEDEX 4 (FR)

(72) Inventor: Jeffrey Earl CHRISTENSEN, Puycasquier (FR)

(21) Appl. No.: 18/971,483

(22) Filed: Dec. 6, 2024

Related U.S. Application Data
(63) Continuation of application No. PCT/IB2023/055841, filed on Jun. 6, 2023.
(60) Provisional application No. 63/365,917, filed on Jun. 6, 2022.

(51) Int. Cl. G06F 16/2457

(52) U.S. Cl. CPC G06F 16/24575

(57) ABSTRACT

The present disclosure relates to techniques for using multi-omics data and artificial intelligence to discover variables correlated with a product target in various domains. Particularly, aspects are directed to a computer implemented method that includes (i) identifying groups of biologically and statistically similar variables, and (ii) selecting, using various artificial intelligence techniques (e.g., machine learning models and rule based systems), groups of variables that are associated with the target feature while taking into account the groups of biologically and statistically similar variables identified in (i). The results of the various artificial intelligence techniques are then cross-referenced to refine and hierarchize a final set of variables associated with the feature of interest.

### CROSS REFERENCE TO RELATED APPLICATIONS

The present application is a continuation of PCT/IB2023/055841, filed Jun. 6, 2023, which claims the benefit and priority of U.S. Provisional Application No. 63/365,917, filed Jun. 6, 2022, the entire contents of which are incorporated herein by reference for all purposes.

### FIELD

The present invention relates to multi-omics based techniques for product target discovery, and in particular, to techniques that use multi-omics data and artificial intelligence to discover variables (e.g., groups of related organisms or operational taxonomic units) correlated with a product target (e.g., low feed conversion ratio, high body mass, etc.) in various domains (e.g., agrofood, pet health, disease, and the like).

### BACKGROUND

Omics refers to a field of study in biological sciences that ends with—omics, such as genomics, transcriptomics, proteomics, epigenomics, metagenomics, metabolomics, or microbiome related multi-omics. The ending -ome is used to address the objects of study of such fields, such as the genome, proteome, transcriptome, epigenome, metagenome, or metabolome, respectively. The genome is the complete sequence of DNA in a cell or organism. The transcriptome is the complete set of RNA transcripts from DNA in a cell or tissue. Bulk and single cell transcriptomes include ribosomal RNA (rRNA), messenger RNA (mRNA), transfer RNA (tRNA), microRNA (miRNA), and other non-coding RNA (ncRNA). The proteome is the complete set of proteins expressed by a cell, tissue, or organism. The proteome is inherently complex because proteins can undergo post-translational modifications (glycosylation, phosphorylation, acetylation, ubiquitylation, and many other modifications to the amino acids comprising proteins), have different spatial configurations and intracellular localizations, and interact with other proteins as well as other molecules. The epigenome is comprised of reversible chemical modifications to DNA and histones, ncRNAs, and the chromatin architecture, wherein interactions or crosstalk between any or all of these epigenetic mechanisms can produce changes in the expression of genes without altering their base sequence. The metabolome is the complete set of small molecule metabolites found within a biological sample (including metabolic intermediates in carbohydrate, lipid, amino acid, nucleic acid, and other biochemical pathways, along with hormones and other signaling molecules, as well as exogenous substances such as drugs and their metabolites). Overall, the objective of omics sciences is to identify, characterize, and quantify all biological molecules that are involved in the structure, function, and dynamics of a cell, tissue, or organism.

Omics-based techniques, including genomics, transcriptomics, proteomics, epigenomics, metagenomics, metabolomics, and bioinformatics, have become recognized as effective tools needed to construct innovative strategies to discover product targets. For example, with respect to drug target discovery, each type of omics data provides important information highlighting differences between normal and abnormal conditions. This data can be utilized to discover diagnostic and prognostic markers and to give insight as to which biological processes are different between the disease and control samples. However, considerable limitations remain. For example, it is notable that any single omics-based method may only view a small portion of the entire picture, which can be insufficient to define precise molecular targets accurately or clearly within complex biochemical and physiological networks. Thus, the complementary or synergistic integration of multiple approaches, at system-wide levels, are warranted. Multi-omics (multiple omics) provides the integrated perspective to power discovery across multiple levels of biology. This biological analysis approach combines genomic data with data from other modalities such as transcriptomics, epigenetics, and proteomics, to measure gene expression, gene activation, and protein levels. Multi-omics profiling studies enable a more comprehensive understanding of molecular changes contributing to normal development, cellular response, and disease. Using integrative omics technologies, researchers can better connect genotype to phenotype and fuel the discovery of novel product targets. Further, strategies that employ integrated multi-omics methodologies can simultaneously clarify, define, and validate multiple potential product targets and action mechanisms for successful candidate development.

### BRIEF SUMMARY

Techniques (e.g., systems, methods, computer program products storing code or instructions executable by one or more processors) disclosed herein relate generally to using a computing system comprising an omics database and an artificial intelligence based discovery platform to discover variables (e.g., groups of related organisms or operational taxonomic units) correlated with a product target (e.g., low feed conversion ratio, high body mass, etc.) in various domains (e.g., agrofood, pet health, disease, and the like). The omics database is provisioned as a two part structure that encompasses biological and in silico components storing data containing numerous variables of different origins and natures including: whole genome sequencing (WGS) metagenomics data, collection of metagenomics assembled genomes (MAGs), inferred metabolic pathways, metabolomics (biochemical data), biological data (e.g., body weight, food intake, feed conversion ratio (FCR), etc.), genetic information (e.g., single nucleotide polymorphisms (SNPs)), and the like. The discovery platform takes as input at least one data set comprising at least two or more types of omics data (private and public biological and/or in silico studies that encompass in vivo, in vitro, and computationally simulated experiments, respectively, that have generated omics samples), that contain numerous variables of different origins and natures, and correlates sets of variables of different natures with a target feature (e.g., a product target) using a two-step approach. The two-step approaches include: (i) identifying groups of biologically and statistically similar variables, and (ii) selecting, using various artificial intelligence techniques (e.g., machine learning models and rule based systems), groups of variables that are associated with the target feature while taking into account the groups of biologically and statistically similar variables identified in (i). The results of the various artificial intelligence techniques are then cross-referenced to refine and hierarchize a final set of variables associated with the feature of interest. The final set of variables is then presented to a client as tables and graphs that describe the significance of the relationship between variables associated with the client's feature of interest.

In various embodiments, a computer-implemented method is provided that includes: receiving a query for a discovery platform configured to generate a final set of variables as an answer to the query where the query comprises key terms and at least one feature of interest, the discovery platform comprises a database and multiple analytical pipelines, the database comprises sets of processed multi-omics data, and each of the multiple analytical pipelines comprises cluster and regression analysis algorithms or models, one or more machine learning models, and a multivariate dimensionality-reduction model; executing the query on the database to retrieve variables discovered to be linked across the sets of multi-omics data that answer the query based on the key terms and the at least one feature of interest; selecting at least one of the multiple analytical pipelines to be used for analyzing the variables based on the at least one feature of interest; analyzing the variables using the at least one of the multiple analytical pipelines to generate the final set of variables as the answer to the query, wherein the analyzing comprises identifying, by the cluster and regression analysis algorithms or models, a first and second set of variables that are associated with the at least one feature of interest that take into account the variables and relationships between the variables; predicting, by the one or more machine learning models, a third set of variables that are associated with the at least one feature of interest that take into account the variables and the relationships between the variables; identifying, by the multivariate dimensionality-reduction model, a fourth set of variables that are associated with the at least one feature of interest that take into account the variables and the relationships between the variable; generating a final set of variables that are correlated with the at least one feature of interest by cross-referencing at least the first set of variables, the second set of variables, the third set of variables, and the fourth set of variables; and outputting the final set of variables as the answer to the query.

In some embodiments, the computer-implemented method further comprises: encoding the variables from the sets of multi-omics data into high dimensional vectors and/or matrices; and generating, by data transformations, normalized and reduced dimensional vectors and/or matrices based on the high dimensional vectors and/or matrices, wherein the analyzing further comprises inputting the normalized and reduced dimensional vectors and/or matrixes into at least one of the multiple analytical pipelines of the discovery platform.

In some embodiments, the computer-implement method further comprises: selecting at least one of the multiple analytical pipelines, which determines whether each of the at least one feature of interest is at least one categorical variable, at least one continuous variable, or a mixture of both; and selecting at least one of the multiple analytical pipelines to be used for analyzing the variables based on each of the at least one feature of interest being at least one categorical variable, at least one continuous variable, or a mixture of both.

In some embodiments, when the at least one feature of interest is a categorical variable the one or more machine learning models comprise a Least Absolute Shrinkage and Selection Operator (LASSO) logit regression model, an elastic net penalized logit regression model where the penalization parameter is an optimized value, and a random forest or random decision forest classifier; and when the at least one feature of interest is at least one continuous variable the one or more machine learning models comprise the LASSO regression model and the elastic net penalized regression model where the penalization parameter is an optimized value.

In some embodiments, when the at least one feature of interest is a categorical variable, the LASSO logit regression model predicts a first and second set of groups of the variables, the elastic net penalized logit regression model where the penalization parameter is an optimized value predicts a third set of groups of the variables, and the random forest or random decision forest classifier predicts a fourth set of groups of the variables, and the first prediction for a set of variables from the LASSO regression model, the second prediction for a set of variables from the elastic net penalized regression model where the penalization parameter is an optimized value, or the third prediction for a set of variables from the random forest or random decision forest classifier are graphed on a receiver operating characteristic curve, and the most robust prediction is selected as the third set of groups of variables.

In some embodiments, when the at least one feature of interest is at least one continuous variable, the LASSO regression model predicts a first and second set of groups of the variables, the elastic net penalize regression model where the penalization parameter is an optimized value predicts a third set of groups of the variables, and the first prediction for a set of variables from the LASSO regression model, the second prediction for a set of variables from the elastic net penalized regression model where the penalization parameter is an optimized value, or the third prediction for a set of variables based on the mean squared error observed for each of the LASSO regression model are graphed on a receiver operating characteristic curve, and the most robust prediction is selected as the third set of groups of variables.

In some embodiments, when the at least one feature of interest is the categorical variable the multivariate dimensionality-reduction model is a sparse partial least square—discriminant analysis model; and when the at least one feature of interest is the at least one continuous variable the multivariate dimensionality-reduction model is a sparse partial least square regression model.

In some embodiments, the sets of multi-omics data are generated by collecting raw data from one or more data repositories and processing the raw data using a data processing pipeline; the raw data relates to any source of multi-omics data such as private and public biological and/or in silico studies that encompass in vivo, in vitro, and computationally simulated experiments, respectively, that have generated the omics samples; and the data processing pipeline comprises bioinformatic tools and a data encoding and transformation system;

In some embodiments, the computer-implemented method further comprises executing the query on the database and determining whether the variables from the sets of multi-omics data answer the query based on the key terms and the at least one feature of interest; and determining whether the variables from the sets of multi-omics answer the query comprise selecting those omics data with relevant biological information towards the query (e.g., if the query is executed on improvements for crop growth, then the relevant omics data may comprise genomic, transcriptomic, and proteomic data) wherein all the biologically relevant data stored in the database is input for the discovery platform.

In some embodiments, the computer-implemented method further comprises selecting at least one of the multiple analytical pipelines to be used for analyzing the variables comprises choosing, either manually or through μl, the multiple analytical pipelines to run based on if the variable is at least one continuous, at least one categorical, or a mixture of both. Optionally, all components of the analytical pipeline may be chosen.

In some embodiments, the computer-implemented method further comprises cross-referencing at least the first set of variables, the second set of variables, the third set of variables, and the fourth set of variables to generate the final set of variables, comprises: choosing variables that convey the same biological information determined by core clustering and the use of biological information from previously known biological pathways.

In some embodiments, the computer-implemented method further comprises outputting the final set of variables rendering the final set of variables in a graphical user interface as an answer to the query wherein the graphical user interface helps to interpret the data and display a selected set of final variables as the product based on the at least one feature of interest.

In some embodiments, the computer-implemented method further comprises manufacturing of at least one product based on the final set of variables wherein the final set of variables comprise a list of variables that when combined have a more significant biological impact compared to when they are alone; wherein the product will comprise of variables absent of false positives and comprised of a combination of variables that optimally perform better together. In some instances, the computer-implemented method provides the foundation for the manufacturer to assemble at least one product in the correct order and proportions based on the different products obtained.

In some embodiments, a system is provided that includes one or more data processors and a non-transitory computer readable medium containing instructions which, when executed on the one or more data processors, cause the one or more data processors to perform part or all of one or more methods or processes disclosed herein.

In some embodiments, a computer-program product is provided that is tangibly embodied in a non-transitory machine-readable medium and that includes instructions configured to cause one or more data processors to perform part or all of one or more methods disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present invention will be better understood in view of the following non-limiting figures, in which:

FIG. 1 shows a framework for discovering unique insights and developing novel solutions for users in accordance with various embodiments;

FIG. 2 shows a workflow for using processed multi-omics data and artificial intelligence to discover variables correlated with a feature of interest in various domains in accordance with various embodiments;

FIG. 3 shows a machine learning model training and deployment system in accordance with various embodiments;

FIG. 4 shows an exemplary flow of a process for collecting and processing any source of multi-omics data such as private and public biological and/or in silico studies that encompass in vivo, in vitro, and computationally simulated experiments, respectively, that have generated the omics samples in accordance with various embodiments; and

FIG. 5 shows an exemplary flow of a process for discovering unique insights to answer user queries in accordance with various embodiments.

### DETAILED DESCRIPTION

### I. Introduction

Target identification and validation in the realm of bio-products or natural products involves identifying biological molecules (e.g., mRNA, nutrients, etc.) and conducting experimental validations to demonstrate an effect (e.g., a therapeutic or dietary effect). In the target identification process, it is important to comprehensively characterize the mechanism of action at the cellular or organism levels because the products may affect a single target, more than one target, and/or simultaneously impact multiple systems within an organism. High-throughput omics methods such as proteomics, genomics, transcriptomics, metabolomics, and bioinformatics-based analysis can provide robust data and have remarkable potential to identify product targets and mechanisms. One major advantage of the use of multi-omics methods is that they are not guided by, or influenced by prior assumptions, making omics a useful tool for identifying and validating previously unknown product targets and revealing novel mechanisms of activation.

Over the past decade many databases and computational platforms for product target discovery have been created to facilitate data mining to identify evidence linking molecules to effects. These tools generally aim to assess the efficacy of product targets and, more recently, their safety aspects. Initially, target product discovery platforms were not built specifically for omics-driven target discovery; however, the platforms have evolved over time to incorporate omics-driven target discovery. For example, target product discovery platforms using genomic, transcriptomics or proteomics data were developed to link targets more efficiently to diseases and to validate these targets. However, these early methods were only able to utilize a single omics data set, limiting the selectivity, specificity, and biochemical or physiological relevance. To address this challenge, efforts have been made towards improving the integration of two or more omics datasets for a more robust analysis of omics-driven target discovery. When combined, multi-omics methods can make full use of advantages in data mining bioinformatics and its integration with artificial intelligence to dramatically enhance the ability to decipher product target associations from off-target effects and non-target associated non-specific binding interactions and other false positives.

Other challenges that remain as critical areas for technological development include: (i) understanding the statistical behavior of readouts from each omics approach independently, (ii) capitalizing on time resolution in omic datasets, such as time course studies, to inform directionality, (iii) interpreting integrated omic datasets given that the variances among samples become large and sparse and render cluster analysis uninformative, (iv) reducing the computational load when analyzing thousands of measurements in each omics experiment to extract meaningful correlations and true interactions and (v) recognizing non-obvious relationships that exist between omic data sets within their original biological context. Currently, there is no single approach for processing, analyzing and interpreting all data from different omic datasets. The need for multimodal data amalgamation strategies and development of reproducible, high throughput, user friendly and effective frameworks must be addressed to fully integrate several of these omics approaches for product target discovery.

To address these challenges and others, various embodiments are directed to a discovery platform and techniques for using multi-omics data and artificial intelligence to discover variables correlated with a product target in various domains. These embodiments include the development of a discovery platform that utilizes multiple analytical pipelines comprising cluster and regression analysis algorithms or models, one or more machine learning models, and a multivariate dimensionality-reduction model to discover obvious and non-obvious relationships that exist between two or more types of omics data based on a client executed query on a feature of interest. Importantly, the workflow described herein provides a single computational pipeline that can collect and process at least two or more omic data sets, analyze and discover relationships between relevant variables within the omics data and a feature of interest, and finally interpret these relationships and provide a client with a final set of variables in a graphical interface as an answer to their query. The integration of at least two or more omic data sets increases the likelihood of discovering novel relations between variables and increases the robustness of the discovery platform, compared to other platforms lacking this feature. Furthermore, because the discovery platform utilizes a targeted approach, a specific collection of references, to discover relationships that exist between omics data, the computational load is reduced. This is further enhanced by the design of the discovery platform to process the multiple analytical pipelines in a highly parallelized manner.

In one particular example, a computer-implemented methos is provided that includes receiving a query for a discovery platform configured to generate a final set of variables as an answer to the query; executing the query on the database to retrieve variables discovered to be linked across the sets of multi-omics data that answer the query based on the key terms and the at least one feature of interest; analyzing the variables using the at least one of the multiple analytical pipelines to generate the final set of variables as the answer to the query, wherein the analyzing comprises identifying, by the cluster and regression analysis algorithms or models, a first and second set of variables that are associated with the at least one feature of interest that take into account the variables and relationships between the variables; predicting, by the one or more machine learning models, a third set of variables that are associated with the at least one feature of interest that take into account the variables and the relationships between the variables; identifying, by the multivariate dimensionality-reduction model, a fourth set of variables that are associated with the at least one feature of interest that take into account the variables and the relationships between the variable; generating a final set of variables that are correlated with the feature(s) of interest by cross-referencing at least the first set of variables, the second set of variables, the third set of variables, and the fourth set of variables; and outputting the final set of variables as the answer to the query.

Advantageously, these techniques allow for the detection of groups of variables that, when put together, will provide a more robust product. This is related to the design of the discovery platform and its ability to incorporate two or more omics data sets into its multiple analytical pipelines to reduce the risk of false positive data, thereby increasing the biological relevance and robustness of the final set of variables comprising the final product. Using this targeted approach reduces the total number of variables that could otherwise be included in the final product and dilutes its overall efficacy. Moreover, the specialized product encourages the reduction of developmental costs and improved market access.

### II. Data Collection, Processing, and Incorporation into Discovery Platform

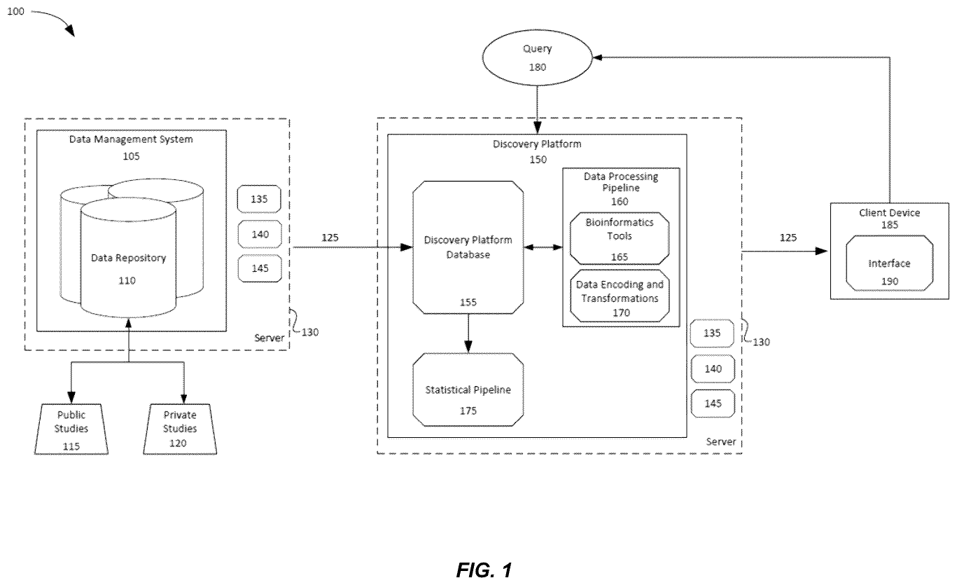

FIG. 1 shows framework 100 for discovering unique insights and developing novel solutions for users in accordance with aspects of the present disclosure. Framework 100 includes a data management system 105, a discovery platform 150, and a client device 185. Although FIG. 1 illustrates a particular arrangement of a data management system 105, a discovery platform 150, and a client device 185, this disclosure contemplates any suitable arrangement of a data management system 105, a discovery platform 150, and a client device 185. As another example, a data management system 105, a discovery platform 150, and two or more client device 185 may be physically or logically co-located with each other in whole or in part. Moreover, although FIG. 1 illustrates a particular number of a data management system 105, a discovery platform 150, and a client device 185, this disclosure contemplates any suitable number of data management systems 105, discovery platforms 150, and client devices 185. As an example, and not by way of limitation, framework 100 may include multiple data management systems 105, discovery platforms 150, and client devices 185.

This disclosure contemplates any type of network familiar to those skilled in the art that may support data communications using any variety of available protocols including without limitation TCP/IP (transmission control protocol/Internet protocol), SNA (systems network architecture), IPX (Internet packet exchange), AppleTalk®, and the like. Merely by way of example, network(s) may be a local area network (LAN), networks based on Ethernet, Token-Ring, a wide-area network (WAN), the Internet, a virtual network, a virtual private network (VPN), an intranet, an extranet, a public switched telephone network (PSTN), an infra-red network, a wireless network (e.g., a network operating under any of the Institute of Electrical and Electronics (IEEE) 1002.11 suite of protocols, Bluetooth®, and/or any other wireless protocol), and/or any combination of these and/or other networks.

Links 125 may connect a data management system 105, a discovery platform 150, and a client device 185 to a network or to each other. This disclosure contemplates any suitable links 157. In particular embodiments, one or more links 125 include one or more wireline (such as for example Digital Subscriber Line (DSL) or Data Over Cable Service Interface Specification (DOCSIS)), wireless (such as for example Wi-Fi or Worldwide Interoperability for Microwave Access (WiMAX)), or optical (such as for example Synchronous Optical Network (SONET) or Synchronous Digital Hierarchy (SDH)) links. In particular embodiments, one or more links 125 each include an ad hoc network, an intranet, an extranet, a VPN, a LAN, a WLAN, a WAN, a WWAN, a MAN, a portion of the Internet, a portion of the PSTN, a cellular technology-based network, a satellite communications technology-based network, another link 125, or a combination of two or more such links 125. Links 125 need not necessarily be the same throughout framework 100. One or more first links 125 may differ in one or more respects from one or more second links 125.

The database management system 105 is a software component that may be executed by one or more processors, hardware components, or combinations thereof in order to control the storage, organization, and retrieval of data (e.g., multi-omics data). The database management system 105 includes one or more data repositories 110, code (e.g., Kernel code) that manages memory and storage, a repository of metadata that includes a collection of database tables and views containing reference information about the database, its structures, and its users.

A data repository 110 is a data storage space for an entity (or sometimes entities) into which data has been specifically partitioned for an analytical or reporting purpose. In some instances, the data relate to any source of multi-omics data such as private 115 and public 120 biological and/or in silico studies that encompass in vivo, in vitro, and computationally simulated experiments, respectively, including multi-omics data such as genomics, proteomics, metabolomics, metagenomics, transcriptomics, etc., variables (e.g., physical information from an individual (size, weight, age), genetic sequences (DNA, RNA), molecule quantities (lipid or protein abundances), information mapping, and the like.

Biological studies (both public studies 115 and private studies 120) provide a large array of data including physical information of a subject (e.g., size, weight, age, etc.), genetic sequences (DNA, RNA, etc.), molecule quantities (e.g., lipid quantity, protein quantity, etc.), and the like. The various data may be acquired separately from individual studies, subjects, and samples, but are in fact related to each other by one or more biological processes. In some instances, the various data include multi-omics data including: genomics, proteomics, metabolomics, metagenomics, and transcriptomics. Multi-omics data provides a complete view of the mechanisms involved in biological processes and allows for better identification of the variables controlling the biological processes to be studied. The data may include variables of different origins and different natures including: whole genome sequencing (WGS) metagenomics data, collection of metagenomics assembled genomes (MAGs), inferred metabolic pathways, metabolomics (biochemical data) from biological materials, biological data (e.g., body weight, food intake, feed conversion ratio (FCR), etc.), genetic information (e.g., single nucleotide polymorphisms (SNPs)), and the like. In certain instances, the data collected from sequencing may include microbial community sequencing data, which can be organized into large relations or matrices where the columns represent samples, and the rows contain observed counts of clustered sequences commonly known as operational taxonomic units, or OTUs, that represent organism types (e.g., OTUs—clustering of any individual animal, plant, or microorganism including bacteria, viruses, parasites, and fungi).

The data repositories 110 may reside in a variety of locations including server 130. For example, a data repository used by server 130 may be local to server 130 or may be remote from server 130 and in communication with server 130 via a network-based or dedicated connection network. In an instance of multiple data repositories such as data repositories 110(a) and 110(b), the data repositories 110(a) and 110(b) may be of different types or of the same type.

A discovery platform 150 comprises a discovery platform database 155, a data processing pipeline 160 that comprises bioinformatic tools 165 and data encoding and transformation process 170, and a statistical pipeline 175 for the purpose of analyzing and visualizing data. The discovery platform 150 is an agnostic platform for multi-omics data analysis (that is, sets of variables of different natures that could be correlated with a target feature). Moreover, the discovery platform 150 can be used to answer a client executed query 180 initiated for a target feature/feature of interest, wherein answering the client executed query 180 comprises retrieving various types of associated data comprising sets of variables and relationships between the variables.

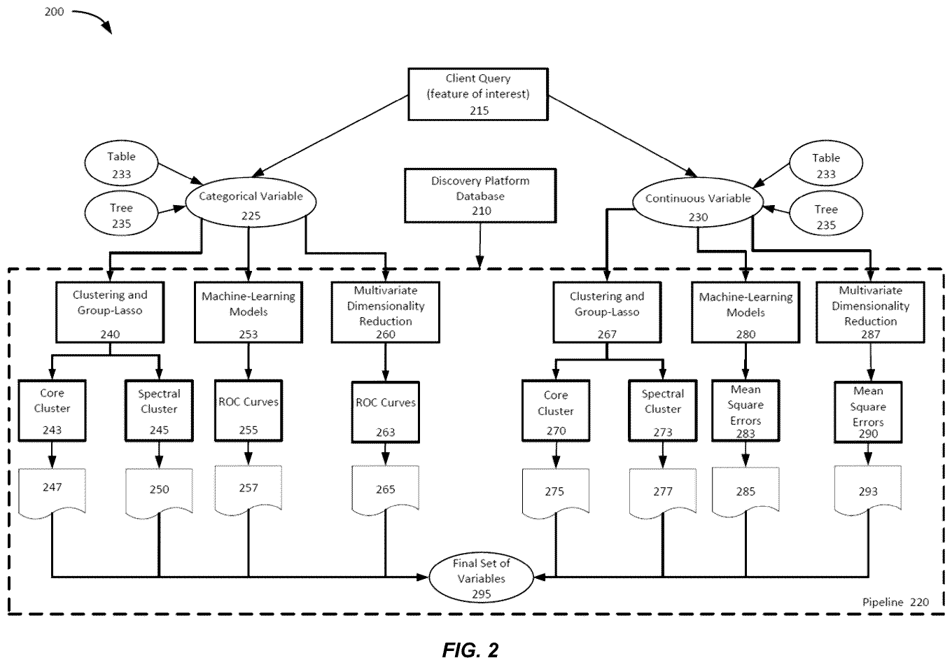

In the configuration depicted in FIG. 1, the discovery platform 150 includes a discovery platform database 155 that receives and stores both raw omics data from the one or more data repositories 110 as well as encoded and transformed data from the data processing pipeline 160, and a statistical pipeline 175 of bioinformatic, statistical, and artificial intelligence-based algorithms and models that implement the functions performed by discovery platform 150 onto the discovery platform database 155. In some instances, the discovery platform 150 is used to: (i) identify groups of biologically and statistically similar variable, (ii) execute various approaches (machine learning approaches, univariate approach, machine learning for group selection, etc.) that take into account this group structure among the variables to select groups of variables that are associated with a target feature such as a product target, and (iii) cross-reference the results of the various approaches to refine and hierarchize the final set of variables associated with the target feature, as described in detail with respect to FIG. 2. The discovery platform 150 may reside in a variety of locations including servers 130. For example, a discovery platform 150 used by server 130 may be local to server 130 or may be remote from server 130 and in communication with server 130 via a network-based or dedicated connection of the network. The discovery platform 150 may be of different configurations or of the same configuration. The one or more servers 130 may be configured to execute a discovery application that provides discovery services to other computer programs or to computing devices (e.g., client device 185) within the computing environment, as defined by a client-server model.

In various instances, server 130 may be adapted to run one or more services or software applications that enable one or more embodiments described in this disclosure. In certain instances, server 130 may also provide other services or software applications that may include non-virtual and virtual environments. In some examples, these services may be offered as web-based or cloud services, such as under a Software as a Service (SaaS) model to the users of client device 185. Users operating client device 185 may in turn utilize one or more client applications to interact with server 130 to utilize the services provided by these components (e.g., database and discovery applications). In the configuration depicted in FIG. 1, server 130 may include one or more components 135, 140 and 145 that implement the functions performed by server 130. These components may include software components that may be executed by one or more processors, hardware components, or combinations thereof. It should be appreciated that different device configurations are possible, which may be different from framework 100. The example shown in FIG. 1 is thus one example of a framework 100 (e.g., a distributed system for implementing an example framework 100) and is not intended to be limiting.

Server 130 may be composed of one or more general purpose computers, specialized server computers (including, by way of example, PC (personal computer) servers, UNIX® servers, mid-range servers, mainframe computers, rack-mounted servers, etc.), server farms, server clusters, or any other appropriate arrangement and/or combination. Server 130 may include one or more virtual machines running virtual operating systems, or other computing architectures involving virtualization such as one or more flexible pools of logical storage devices that may be virtualized to maintain virtual storage devices for the server. In various instances, server 130 may be adapted to run one or more services or software applications that provide the functionality described in the foregoing disclosure.

The computing systems in server 130 may run one or more operating systems including any of those discussed above, as well as any commercially available server operating system. Server 130 may also run any of a variety of additional server applications and/or mid-tier applications, including HTTP (hypertext transport protocol) servers, FTP (file transfer protocol) servers, CGI (common gateway interface) servers, JAVA® servers, database servers, and the like. Exemplary database servers include without limitation those commercially available from Oracle®, Microsoft®, Sybase®, IBM® (International Business Machines), and the like.

In some implementations, server 130 may include one or more applications to analyze and consolidate data feeds and/or data updates received from users of client computing devices 180. As an example, data feeds and/or data updates may include, but are not limited to, in vivo feeds, in silico feeds, or real-time updates received from public studies, user studies, one or more third party information sources, and data streams (continuous, batch, or periodic), which may include real-time events related to sensor data applications, biological system monitoring, and the like. Server 130 may also include one or more applications to display the data feeds, data updates, and/or real-time events via one or more display devices of client computing devices 185.

The data processing pipeline 160 is a software component that may be executed by one or more processors, hardware components, or combinations thereof in order to control the organization and structure of data from the discovery platform database 155 (e.g., multi-omics data). The data processing pipeline 160 includes bioinformatics tools 165 and data encoding and transformation methods 170.

Bioinformatic tools 165 can comprise various computational tools used for assembling, annotating, aligning/mapping, profiling, etc., the omics data stored in the discovery platform database 155. In some embodiments, one or more processing steps comprise assembling raw sequencing reads to build a metagenomic assembly genome (MAG). As used herein, “reads” (e.g., “a read,” “a sequence read”) are short nucleotide sequences produced by any sequencing process known in the art. Reads can be generated from one end of nucleic acid fragments (“single-end reads”), and sometimes are generated from both ends of nucleic acid fragments (e.g., paired-end reads, double-end reads). A MAG comprises combined genomic DNA of an entire environment of samples representing the microbial genomes that have been processed by computational metagenomic assemblers.

In some embodiments, genomic annotation can be performed to determine where components (e.g., genes, regulatory elements, and the like) in a genome are located and/or to determine the function of the components in the genome.

In some embodiments, aligning and mapping sequence reads to a specified nucleic acid region (e.g., a chromosome or portion thereof) and then counting those reads that align to a specific nucleic acid region (referred to as read count). The terms “aligned,” “alignment,” or “aligning” generally refer to two or more nucleic acid sequences and/or amino acids sequences that can be identified as a match (e.g., 100% identity) or partial match. Alignments can be done manually or by a computer (e.g., a software, program, subsystem, or algorithm). Mapping nucleotide sequence reads (i.e., sequence information from a fragment whose physical genomic position is unknown) can be performed in a number of ways, and often comprises alignment of the obtained sequence reads with a matching sequence in a reference genome. In such alignments, sequence reads generally are aligned to a reference sequence and those that align are designated as being “mapped,” as “a mapped sequence read” or as “a mapped read.” Any suitable mapping method (e.g., process, algorithm, program, software, subsystem, the like or combination thereof) can be used. Non-limiting examples of computer algorithms that can be used to align sequences include, without limitation, BLAST, BLITZ, FASTA, BOWTIE 1, BOWTIE 2, ELAND, MAQ, PROBEMATCH, SOAP, BWA or SEQMAP, or variations thereof or combinations thereof. In some embodiments, sequence reads can be found and/or aligned with sequences in nucleic acid databases known in the art including, for example, GenBank, dbEST, dbSTS, EMBL (European Molecular Biology Laboratory) and DDBJ (DNA Databank of Japan).

Mapped sequence reads that have been counted are referred to herein as raw data, since the data represents unmanipulated counts (e.g., raw counts). In some embodiments, sequence read data in a data set can be processed further (e.g., mathematically and/or statistically manipulated) and/or displayed to facilitate providing an outcome. In certain embodiments, data sets, including larger data sets, may benefit from pre-processing to facilitate further analysis. Processing of data sets sometimes involves removal of redundant and/or uninformative portions or portions of a reference genome (e.g., portions of a reference genome with uninformative data, redundant mapped reads, portions with zero median counts, overrepresented or underrepresented sequences). Without being limited by theory, data processing and/or preprocessing may (i) remove noisy data, (ii) remove uninformative data, (iii) remove redundant data, (iv) reduce the complexity of larger data sets, and/or (v) facilitate transformation of the data from one form into one or more other forms. Processing can render data more amenable to further analysis and can generate an outcome in some embodiments. In some embodiments one or more or all processing methods (e.g., normalization methods, portion filtering, mapping, validation, the like or combinations thereof) are performed by a processor, a microprocessor, a computer, in conjunction with memory and/or by a microprocessor controlled apparatus.

In some embodiments, the output of the bioinformatic processing comprises tables of samples (columns), features (rows), and the relation between each sample and row (i.e. gene expression, read count, and the like).

Encoding and data transformations 170, are computer implemented methods that convert the sets of variables and the relationships between the variables into high dimensional vectors and/or matrices; and generating, by data transformations, normalized and reduced dimensional vectors and/or matrices based on the high dimensional vectors and/or matrices, where the normalized and reduced dimensional vectors and/or matrixes can be input into the discovery platform database. A vector is a sequence of n numbers each of which is indexed by its position in the sequence. Given some number m of objects, each of which is described by an n-component vector, the set of vectors may be organized as an m×n matrix.

The descriptions of the variables and relationships between them are translated into vectors and matrices using one or more encoding processes such as one-hot encoding, term frequency-inverse document frequency (TF-IDF), Word2Vec, FastText, and the like, which may be implemented using pre-trained embedding models. A one-hot encoding is a representation of categorical variables as binary vectors. Each integer value is represented as a binary vector that is all zero values except for the index of the integer, which is marked with a 1. TF-IDF is a statistical measure used to determine the mathematical significance of words in documents. The vectorization process is similar to one hot encoding. Alternatively, the value corresponding to the word is assigned a TF-IDF value instead of 1. The TF-IDF value is obtained by multiplying the TF and IDF values. In Word2Vec the entire corpus is scanned, and the vector creation process is performed by determining which words the target word occurs with more often. In this way, the semantic closeness of the words to each other is also revealed. The working logic of FastText algorithm is similar to Word2Vec, but the biggest difference is that it also uses N-grams of words during training. This gives the model the ability to predict different variations of words.

In some embodiments, the discovery platform database 155 may comprise of at least two or more omics data sets encoded into tables that may comprise of information about an organism in a genomics table, a transcriptomics table, and a proteomics table, wherein the tables comprise attributes that are shared amongst all the tables, forming relations. In some instances, the discovery platform database 155 may be a relational database. A relational database is a database that conforms to the relational model and the mathematical principles of the relational model define how the discovery platform database 155 should function. The relational model comprises the following aspects: structures, which are well defined objects that store or access the data of the database, operations, which are clearly defined actions that enable applications to manipulate the data and structures of the database, and integrity rules, which govern operations on the data and structures of the database.

The structures of a relational database (as defined by the relational model) are tables, columns (or fields), rows (or records), and keys. A table is a two-dimensional representation of a relation in the form of rows (tuples) and columns (attributes). Each row in a table has the same set of columns. A relational database stores data in a set of simple relations (tables). A relation is a set of tuples (rows). A tuple is an unordered set of attribute values (columns). For example, a relational database could store information about an organism in a genomics table, a transcriptomics table, and a proteomics table. A tuple or row is a single occurrence of the data contained in the table and each row is treated as a single unit. The rows (or records) are organized as a set of columns (or fields). All rows in a table comprise the same set of columns. There are two types of keys: primary and foreign. A primary key is a column (or group of columns) whose value uniquely identifies each row in a table. Because the key value is always unique, the key value can be used to detect and prevent duplicate rows. A foreign key is a column value in one table that is required to match the column value of the primary key in another table. In other words, it is the reference from one table to another. If the foreign key value is not null, then the primary key value in the referenced table must exist. It is this relationship of a column in one table to a column in another table that provides the relational database with its ability to join tables. In some embodiments, the tables comprising information about an organism in a genomics table, a transcriptomics table, and a proteomics table may be encoded into the discovery platform database 155 as defined by the primary/foreign keys, wherein the primary/foreign keys extract out information that is common across the omic data being compared, updating the discovery platform 150.

At block 170, data transformations are applied to the high dimensional vectors and/or matrices. The data transformations may include relative abundance normalization, reference-based transformations, and dimensional reduction. Relative abundance refers to the evenness of distribution of individuals among species in a community or sample. For differential abundance testing between groups (e.g., case vs. control), an approach can be taken to first rarify the count matrix to a fixed depth and then apply a nonparametric test (e.g., the Mann-Whitney/Wilcoxon rank-sum test for tests of two groups; the Kruskal-Wallis test for tests of multiple groups) or parametric test (e.g., parametric models composed of a generalized linear model (GLM)). Normalization enables clustering of samples according to certain factors such as biological origin when the groups differ substantially in their overall composition such as microbial composition. Normalization is the process of transforming the data to enable an accurate comparison of statistics from different measurements by eliminating artifactual biases in the original measurements. For example, in microbiome data, biases that reflect no true difference in underlying biology can exist due to variations in sample collection, library preparation, and/or sequencing and can manifest as, e.g., uneven sampling depth and sparsity. Normalization approaches include (i) rarefying or drawing without replacement from each sample such that all samples have the same number of total read counts. (ii) scaling, which refers to multiplying the matrix counts by fixed values or proportions, i.e., scale factors and specific effects of scaling methods, depend on the scaling factors chosen and how they are applied, and (iii) Aitchison's log-ratio transformation, which is applicable to compositional data, and the like.

Moreover, all components in a composition are mutually dependent features that cannot be understood in isolation. Therefore, any analysis of individual components from data encoding and transformations 170 may be performed with respect to a reference. This reference transforms each sample into an unbounded space where any statistical method can be used. The centered log-ratio (CLR) may be used for this transformation, which uses the geometric mean of the sample vector as the reference. Alternatively, the additive log-ratio (ALR) may be used for this transformation, which uses a single component as the reference. Other transformations use specialized references based on the geometric mean of a subset of components (collectively called multi-additive log-ratio [MALR]transformations). One MALR transformation is the inter-quartile log-ratio (IQLR) transformation, which uses components in the interquartile range of variance. Another transformation that may be used is the robust centered log-ratio (RCLR) transformation, which only uses the non-zero components. After effective normalization and transformation, data from different samples can then be compared to each other in downstream processes.

High-dimensional data can be difficult to interpret. One approach to simplification is to assume that the data of interest lies within a lower-dimensional space, thus allowing the data to be visualized in the low-dimensional space. There are several techniques both nonlinear and linear for dimensionality reduction that can be used alone or in combination to reduce the dimensionality of the high dimensional vectors and/or matrices. These techniques include, without limitation, principal component analysis (PCA), independent component analysis, Laplacian eigenmaps, isomaps, locally-linear embedding, singular value decomposition, Gaussian process latent variable models, t-distributed Stochastic Neighbor Embedding (t-SNE), contagion maps, nonlinear PCA, factor analysis, manifold sculpting, and the like. In some instances, t-SNE is applied to reduce the dimensionality of the high dimensional vectors and/or matrices. t-SNE is an unsupervised non-linear dimensionality reduction and data visualization technique, which embeds the points from a higher dimension to a lower dimension trying to preserve the neighborhood or local structure of that point. The t-SNE algorithm computes the probability that pairs of data points in the high-dimensional space are related, and then chooses low-dimensional embeddings which produce a similar distribution.

Once the data transformations are completed, the reduced dimensional vectors and/or matrices (e.g., normalized OTU tables) can be used in downstream processing (input into the discovery platform database) that identifies sets of variables of different natures from the reduced dimensional vectors and/or matrices that correlate with the feature of interest.

A client device 185 is an electronic device including hardware, software, or embedded logic components or a combination of two or more such components and capable of interacting with the output from the discovery platform 150 with respect to appropriate product target discovery functionalities in accordance with techniques of the disclosure. The client devices 185 may include various types of computing systems such as portable handheld devices, general purpose computers such as personal computers and laptops, workstation computers, wearable devices, gaming systems, thin clients, various messaging devices, sensors or other sensing devices, and the like. These computing devices may run various types and versions of software applications and operating systems (e.g., Microsoft Windows®, Apple Macintosh®, UNIX® or UNIX-like operating systems, Linux or Linux-like operating systems such as Google Chrome™ OS) including various mobile operating systems (e.g., Microsoft Windows Mobile®, iOS®, Windows Phone®, Android™, BlackBerry®, Palm OS®). Portable handheld devices may include cellular phones, smartphones, (e.g., an iPhone), tablets (e.g., iPad®), personal digital assistants (PDAs), and the like. Wearable devices may include Google Glass® head mounted display, and other devices. The client device 185 may be capable of executing different applications such as various Internet-related apps, communication applications (e.g., E-mail applications, short message service (SMS) applications) and may use various communication protocols. This disclosure contemplates any suitable client device 185 configured to generate and output product target discovery content to a user. For example, users may use client device 185 to execute one or more applications, which may generate one or more discovery or storage requests that may then be serviced in accordance with the teachings of this disclosure. A client device 185 allows access to an interface 190 (e.g., a graphical user interface) that enables a user of the client device 185 to interact with the client device 185. The client device 185 may also output information to the user via this interface 190. Although FIG. 1 depicts only one client device 185, any number of client devices 185 may be supported.

### III. Workflow

FIG. 2 shows a workflow 200 for using multi-omics data and artificial intelligence to discover variables correlated with a feature of interest in various domains in accordance with aspects of the present disclosure. Workflow 200 includes a discovery platform database 210, a client executed query 215 conducted for a feature of interest, and a pipeline 220 (e.g., the discovery platform statistical pipeline 175 described with respect to FIG. 1) of bioinformatic, statistical, and artificial intelligence-based algorithms and models configured and trained to identify sets of variables of multiple nature (various domains) that are associated with a feature of interest, e.g., a commercial need or interest such as a product target. As an example, workflow 200 can be used to identify sets of organisms (i.e., any individual animal, plant, or microorganism including bacteria, viruses, parasites, and fungi) and/or molecules (i.e., any group of two or more atoms) associated with a biological performance such as body weight or food conversion rate. The sets of bacteria and/or molecules can then be selected for a final direct fed microbial product configured to achieve a given body weight or food conversion rate.

The conventional approach for selecting organisms/molecules associated with a target feature is to perform univariate or multivariate tests of association between the feature of interest and each of the input variables individually. More recent research uses machine learning algorithms (and more specifically supervised learning approaches) to identify the additive effect of several input variables. Nonetheless, most of the conventional solutions in the industry propose an outcome with too many variables associated with a feature of interest. Therefore, the level of refinement is too low. There is then a need to learn through machine learning approaches from each round of data implementation to eliminate non-informative variables, thereby selecting the most refined variables and hierarchizing them. Hence, during validation (reduction to practice experiments) the number of positive variables is limited and of a higher confidence interval.

To overcome these challenges, the workflow 200 begins with the data stored in the discovery platform database 210. This data contains numerous variables of different origins and natures. The pipeline 220 correlates sets of variables of different natures with a client executed query 215 detailing a feature of interest (e.g., a product target) using a two-step approach. The two-step approaches include: (i) identifying groups of biologically and statistically similar variables (i.e. categorical or continuous variables), and (ii) selecting, using various artificial intelligence techniques (e.g., machine learning models and rule-based systems (clustering, Group-LASSO, and multivariate dimensionality reduction)), groups of variables that are associated with the target feature while taking into account the groups of biologically and statistically similar variables identified in (i). The results of the various artificial intelligence techniques are then cross-referenced to refine and hierarchize a final set of variables associated with the feature of interest. The group selection approach allows for the identification of highly refined groups of similar variables such as organisms (or others like molecules) which when put together will provide better performances (like growth). Advantageously, identifying multiple variables of different natures through multi-omics analyses provides the best view of the mechanisms involved in a biological process and allows better identification of variables controlling the biological process to be studied.

The workflow 200 starts with the encoded/transformed data from the discovery database 210.

The client executed query 215 can be provided as a cloud service to provide additional data storage and computing power to the client. Furthermore, clients will have access to the data stored on the discovery platform database, allowing them to navigate through all the omics data and select the data most relevant to their query.

A client executed query 215 conducted for at least one feature of interest determines whether the feature(s) of interest is at least one categorical 225, at least one continuous 230 variable, or a mixture of both. Categorical variables contain a finite number of categories or distinct groups (may as well be infinite but defined as groups i.e., categories). These variables are either nominal (no natural ordering) or ordinal (ordered categories). For example, categorical variables include gender, race, and age group. Continuous variables are numeric variables that have an infinite number of values between any two values. Continuous variables can be numeric or date/time. For example, continuous variables include an analytical chemistry level, the height or weight of a subject, or the pulse, heart rate, food conversion rate, or respiration rate of a subject. Once the variable type(s) is determined, the processed omics data stored in the discovery platform database 210 is used in discovery platform pipeline 220 for downstream analysis. The bioinformatic, statistical, and artificial intelligence-based algorithms and models used in the downstream processing to identify sets of variables are different based on whether the feature of interest is at least one categorical 225, at least one continuous 230 variable, or a mixture of both.

In the instances that the feature of interest is at least one categorical variable 225, the pipeline 220 also takes into account optional supplemental information that is obtained and appended to the reduced dimensional vectors and/or matrices. The supplemental information can be any data obtainable from the one or more data repositories 110 or third-party sources that can be used to further understand relationships between the dependent variable (feature of interest) and the independent variables and correlations therebetween (variables and relationships within the reduced dimensional vectors and/or matrixes). For example, with respect to OTU tables of variables and a target feature of food conversion rate, the supplemental information obtained may include a taxonomy table 235 and a phylogenetic tree 240. The reduced dimensional vectors and/or matrices and the optional supplemental information are then input into various artificial intelligence-based systems (e.g., machine learning models and rule-based systems) for selecting groups of variables that are associated with the feature of interest while taking into account the groups of biologically and statistically similar variables.

At block 240, the reduced dimensional vectors and/or matrices and the optional supplemental information are input into a clustering and Group Least Absolute Shrinkage and Selection Operator (LASSO) system. More specifically, this system performs core clustering (block 243), or spectral clustering (block 245) followed by Group-LASSO. At block 243, core clustering is performed which comprises the detection of representative variables in dimensional spaces with a potentially limited number of observations. Detection of sets of variables is based on an original graph clustering strategy denoted CORE-clustering algorithm, which detects CORE-clusters, i.e., sets of variables having a user-defined minimal size and in which each variable is very similar to at least another variable. Representative variables are then robustly estimated as the CORE-cluster centers. The core clustering allows for the system to infer groups within the variables. Thereafter, Group-LASSO is applied to detected sets of variables (clusters), which is a regularization technique (between L1 (LASSO) and L2 (Ridge), allowing predefined groups of covariates to be jointly selected into or out of the model. The Group-LASSO ensures that all the variables of the same CORE-cluster encoding the at least one categorical covariate are included or excluded together. At block 247, associated groups of variables (e.g., groups of OTUs) selected by the Group LASSO—(e.g., groups of microbiomes within a host—does not follow Gaussian curve) are output as the groups of variables that are associated with the feature of interest.

At block 245, spectral clustering is performed which is a connectivity approach to clustering, where communities of nodes (i.e., data points) that are connected or immediately next to each other are identified in a graph. The nodes are then mapped to a low-dimensional space that can be easily segregated to form clusters. Spectral clustering uses information from the eigenvalues (spectrum) of special matrices (i.e., Affinity Matrix, Degree Matrix and Laplacian Matrix) derived from the graph or the reduced dimensional vectors and/or matrices. Thereafter, Group-LASSO is applied to detect sets of variables (clusters), which allows predefined groups of covariates to be jointly selected into or out of the model. The Group-LASSO ensures that all the variables of the same spectral-cluster encoding the at least one categorical covariate are included or excluded together. At block 250, associated groups of variables (e.g., groups of OTUs) selected by the Group LASSO—(e.g., groups of microbiomes within a host—does not follow Gaussian curve) are output as the groups of variables that are associated with the feature of interest.

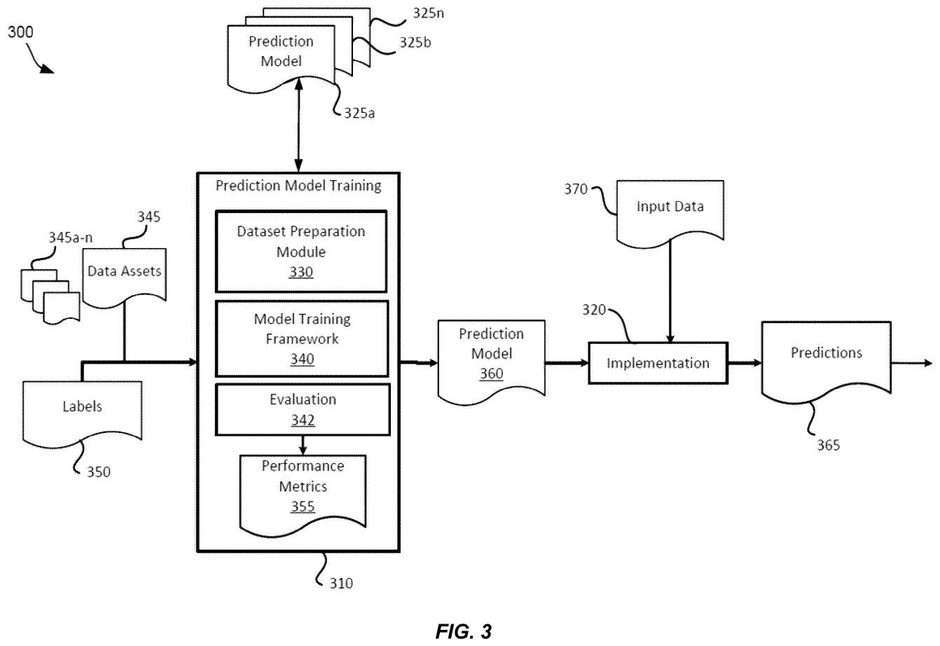

At block 253, the reduced dimensional vectors and/or matrices and the optional supplemental information are input into a machine learning system, as described in detail with respect to FIG. 3. More specifically, this system uses one or more machine learning models to identify groups of variables that are associated with the feature of interest. The one or more machine learning models include a LASSO regression model, an elastic net penalized regression model (where the penalization parameter is 0.2), an elastic net penalized regression model (where the penalization parameter is 0.8), and a random forest or random decision forest classifier. Associated groups of variables (e.g., groups of OTUs) selected or inferred by each of machine learning models are output as the groups of variables that are associated with the feature of interest. A receiver operating characteristic curve (ROC curve), is then observed at block 255 to select at block 257 associated groups of variables (e.g., groups of OTUs) selected or inferred by the best machine learning model or approach (e.g., the model or approach with >80% robustness). The ROC curve is a graphical plot that illustrates the diagnostic ability of a binary classifier system as its discrimination threshold is varied. The ROC curve is created by plotting the true positive rate (TPR) against the false positive rate (FPR) at various threshold settings. The true-positive rate is also known as sensitivity, recall or probability of detection. The false-positive rate is also known as probability of false alarm and can be calculated as (1—specificity). The ROC curve is thus the sensitivity or recall as a function of fall-out (i.e., robustness).

At block 260, the reduced dimensional vectors and/or matrices and the optional supplemental information are input for a multivariate dimensionality-reduction model. More specifically, this system uses sparse partial least squares—discriminant analysis (sPLS-DA) to identify groups of variables that are associated with the feature of interest. sPLS-DA is based on Partial Least Squares regression (PLS) for discrimination analysis, but a LASSO penalization has been added to select variables and the response vector contains at least one categorical vectors rather than continuous vectors. sPLS-DA enables the selection of the most predictive or discriminative features in the data to classify samples or groups of variables. Associated groups of variables (e.g., groups of OTUs) selected by sPLS-DA are output as the groups of variables that are associated with the feature of interest. A ROC curve is then observed at block 263 to select at block 265 associated groups of variables (e.g., groups of OTUs) classified best by the sPLS-DA approach (e.g., groups classified with >80% robustness).

In the instances that the feature of interest is at least one continuous variable 230, the pipeline 220 continues where optional supplemental information is obtained and appended to the reduced dimensional vectors and/or matrices. The supplemental information can be any data obtainable from the one or more data repositories 110 or third-party sources that can be used to further understand relationships between the dependent variable (feature of interest) and the independent variables and correlations between them (variables and relationships within the reduced dimensional vectors and/or matrices). The reduced dimensional vectors and/or matrices and the optional supplemental information are then input into various artificial intelligence-based systems (e.g., machine learning models and rule-based systems) for selecting groups of variables that are associated with the feature of interest while taking into account the groups of biologically and statistically similar variables. In some instances, the artificial intelligence-based systems used for at least one continuous variable are the same as the artificial intelligence-based systems used for at least one categorical variable, and thus the details of such systems are not repeated herein for purposes of brevity.

At block 267, the reduced dimensional vectors and/or matrices and the optional supplemental information are input into a clustering and Group-LASSO system. More specifically, this system performs core clustering (block 270) or spectral clustering (block 273) followed by Group-LASSO. At blocks 275 (first set of groups) and 277 (second set of groups), associated groups of variables (e.g., groups of OTUs) selected by the Group LASSO—(e.g., groups of microbiomes within a host—does not follow Gaussian curve) are output as the groups of variables that are associated with the feature of interest.

At block 280, the reduced dimensional vectors and/or matrices and the optional supplemental information are input into a machine learning system, as described in detail with respect to FIG. 3. More specifically, this system uses one or more machine learning models to identify groups of variables that are associated with the feature of interest. The one or more machine learning models include a LASSO regression model, an elastic net penalized regression model (where the penalization parameter is 0.2), and an elastic net penalized regression model (where the penalization parameter is 0.8). Associated groups of variables (e.g., groups of OTUs) are selected or inferred by each of machine learning models are output as the groups of variables that are associated with the feature of interest. A mean squared error (MSE) is then observed at block 283 to select at block 285 (third set of groups). Associated groups of variables (e.g., groups of OTUs) are selected or inferred by the best machine learning model or approach (e.g., the model or approach with >80% robustness). The (MSE or mean squared deviation (MSD) of a predictor such as the machine learning models measures the average of the squares of the errors—that is, the average squared difference between the predicted values and the actual value. Thus, the MSE assesses the quality or robustness of a predictor (i.e., a function mapping arbitrary inputs to a sample of values of some random variable).

At block 287, the reduced dimensional vectors and/or matrices and the optional supplemental information are input into a multivariate dimensionality-reduction model. More specifically, this system uses sparse partial least squares (sPLS) to identify groups of variables that are associated with the feature of interest. PLS regression reduces the number of variables by projecting independent variables onto latent structures. sPLS combines variable selection and modeling in a one-step procedure. This is done by including the LASSO penalization on loading vectors to reduce the number of original variables used when constructing latent variables. Associated groups of variables (e.g., groups of OTUs) selected by sPLS are output as the groups of variables that are associated with the feature of interest. A MSE is then observed at block 290 to select at block 293 associated groups of variables (e.g., groups of OTUs) classified best by the sPLS approach (e.g., groups classified with >80% robustness).

At block 295, the results of the various artificial intelligence techniques (blocks 247, 250, 257, and 265, and/or 275, 277, 285, and 293) are then cross-referenced to refine and hierarchize a final set of variables associated with the feature of interest. The group selection approach allows for the identification of highly refined groups of similar variables such as organisms (or others like molecules) which when put together will provide better performances.

### IV. Discovery Platform: Machine Learning

Various embodiments also relate to using artificial intelligence (rule based or machine learning) to predict associated groups of variables corresponding to a feature of interest. FIG. 3 illustrates a machine learning model training and deployment system 300 in accordance with some embodiments. The machine learning model training and deployment system 300 may be a component in a discovery platform (e.g., discovery platform 175 described with respect to FIG. 1). As shown in FIG. 3, the machine learning model training and deployment system 300 includes various stages: a prediction model training stage 310 to build and train models, an evaluation stage 342 to evaluate performance of trained models, and an implementation stage 320 for implementing one or more models. The prediction model training stage 310 builds and trains one or more prediction models 325a-325n (which may be referred to herein individually as a prediction model 325 or collectively as the prediction models 325 and ‘n’ represents any natural number) to be used by the other stages. For example, the prediction models 325 can include a model for predicting associated groups of variables corresponding to at least one continuous variable (e.g., feature of interest), a model for predicting associated groups of variables corresponding to at least one categorical variable (e.g., feature of interest), and a model for predicting associated groups of variables corresponding to either continuous and categorical variables or a mixture of both. Still other types of prediction models may be implemented in other examples according to this disclosure.

A prediction model 325 can be a machine learning model, such as a convolutional neural network (“CNN”), e.g., an inception neural network, a residual neural network (“Resnet”), or a recurrent neural network, e.g., long short-term memory (“LSTM”) models or gated recurrent units (“GRUs”) models, other variants of Deep Neural Networks (“DNN”) (e.g., a multi-label n-binary DNN classifier or multi-class DNN classifier). A prediction model 425 can also be any other suitable ML model trained for predicting associated groups of variables, such as a LASSO regression model, an elastic net penalized regression model (where the penalization parameter is 0.2), an elastic net penalized regression model (where the penalization parameter is 0.8), a random forest or random decision forest classifier, a Generative adversarial network (GAN), Naive Bayes Classifier, Linear Classifier, Support Vector Machine, Bagging Models such as random forest or random decision forest classifier, Boosting Models, Extreme Gradient boosting models, Shallow Neural Networks, or combinations of one or more of such techniques—e.g., CNN-HMM or MCNN (Multi-Scale Convolutional Neural Network). The machine learning model training and deployment 300 may employ the same type of prediction model or different types of prediction models for predicting associated groups of variables. Still other types of prediction models may be implemented in other examples according to this disclosure.

To train the various prediction models 325, the training stage 310 consists of two main components: dataset preparation module 330 and model training framework 340. The dataset preparation module 330 performs the processes of loading data assets 345, splitting the data assets 345 into training and validation sets 345a-n so that the system can train and test the prediction models 325, and pre-processing of data assets 345. Splitting the data assets 345 into training and validation sets 345 a-n may be performed randomly (e.g., a 90/10%, 70/30%, or any other appropriate splitting) or the splitting may be performed in accordance with a more complex validation technique such as K-Fold Cross-Validation, Leave-one-out Cross-Validation, Leave-one-group-out Cross-Validation, Nested Cross-Validation, or the like to minimize sampling bias and overfitting.

The training data 345a may include at least a subset of data (e.g., omics data from public and private biological and/or in silico studies) received via a client system and/or obtained from one or more data repositories. The subset of the data can be obtained in various ways including text, audio, images, videos, sensor data, or the like. For example, if the subset of data is provided as images, the data preparation 330 may convert the images to text using an image-to-text converter (not shown) that performs text recognition (e.g., optical character recognition) to determine the text within the image. Additionally or alternatively, the data preparation module 330 may standardize the format of a subset of data. In some instances, the subset of data is provided by a different user or a third party from that of the user involved with training the model and/or using the model in an inference phase. The training data 345a for a prediction model 325 may include the subset of data and labels 350 corresponding to the subset of data as a matrix or table of values. For example, for each example of data, an associated variable to be inferred by the prediction model 325 may be provided as ground truth information for labels 350. The behavior of the prediction model 325 can then be adapted (e.g., through MinMax or Alternating Least Square optimization or Gradient Descent) to minimize the difference between the generated inferences for various variables and the ground truth information.

The model training framework 340 performs the processes of determining hyperparameters for the model 325 and performing iterative operations of inputting examples from the training data 345a into the prediction model 325 to find a set of model parameters (e.g., weights and/or biases) that minimizes a cost function(s) such as loss or error function for the model 325. The hyperparameters are settings that can be tuned or optimized to control the behavior of the model 325. Most models explicitly define hyperparameters that control different features of the models such as memory or cost of execution. However, additional hyperparameters may be defined to adapt the prediction model 325 to a specific scenario. For example, the hyperparameters may include regularization weight or strength. The cost function can be constructed to measure the difference between the outputs inferred using the models 345 and the ground truth annotated to the samples using the labels 350. For example, for a supervised learning-based model, the goal of the training is to learn a function “h( )” (also sometimes referred to as the hypothesis function) that maps the training input space X to the target value space Y, h: X→Y, such that h(x) is a good predictor for the corresponding value of Y. Various different techniques may be used to learn this hypothesis function. In some techniques, as part of deriving the hypothesis function, the cost or loss function may be defined to measure the difference between the ground truth value for an input and the predicted value for that input. As part of training, techniques such as back propagation, random feedback, Direct Feedback Alignment (DFA), Indirect Feedback Alignment (IFA), Hebbian learning, and the like are used to minimize this cost or loss function.

Once the set of model parameters are identified, the model 325 has been trained and the model training framework 340 performs the additional processes of testing or validation using the subset of testing data 345b (testing or validation data set). The testing or validation processes include iterative operations of inputting examples from the subset of testing data 345b into the model 325 using a validation technique such as K-Fold Cross-Validation, Leave-one-out Cross-Validation, Leave-one-group-out Cross-Validation, Nested Cross-Validation, or the like to tune the hyperparameters and ultimately find the optimal set of hyperparameters. Once the optimal set of hyperparameters are obtained, a reserved test set from the subset of test data 345a may be input into the model 325 to obtain output (in this example, one or more groups of variables), and the output is evaluated versus ground truth groups of variables using correlation techniques such as Bland-Altman method and the Spearman's rank correlation coefficients. Further, performance metrics 355 may be calculated in evaluation stage 342 such as the error, accuracy, precision, recall, ROC, etc. The performance metrics 355 may be used in the evaluation stage 342 to analyze performance of the model 325 for predicting associated groups of variables.

The model training stage 310 outputs trained models including one or more trained prediction models 360. The one or more trained prediction models 355 may be deployed and used in the implementation stage 320 to predict associated groups of variables 365 corresponding to a feature of interest such as a product target. For example, prediction models 360 may receive input data 370 (e.g., omics data), and predict groups of variables based on features and relationships between features extracted from within the input data 370.

### V. Techniques of Discovery

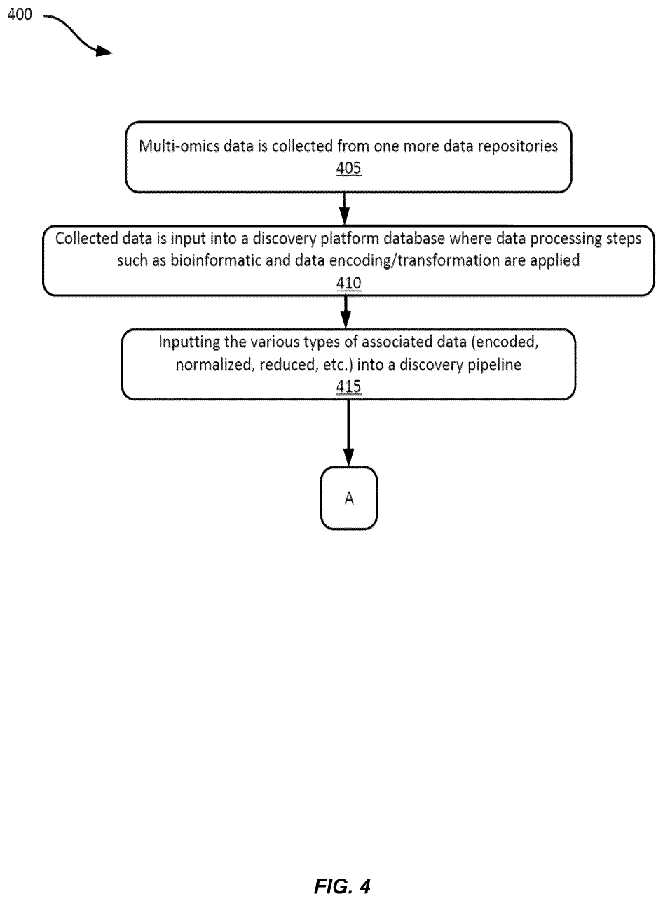

FIG. 4 is a flowchart illustrating a process 400 for collecting and processing multi-omics data related to any source of multi-omics data such as private and public biological and/or in silico studies that encompass in vivo, in vitro, and computationally simulated experiments, respectively, that have generated the omics samples. The processing depicted in FIG. 4 may be implemented in software (e.g., code, instructions, or programing) executed by one or more processing units (e.g., processors or cores) of the respective systems, hardware, or combinations thereof. The software may be stored on a non-transitory storage medium (e.g., on a memory device). The method presented in FIG. 4 and described below is intended to be illustrative and non-limiting. Although FIG. 4 depicts the various processing steps occurring in a particular sequence or order, this is not intended to be limiting. In certain alternative embodiments, the steps may be performed in a different order, or some steps may also be performed in parallel. In certain embodiments, such as in the embodiments depicted in FIGS. 1-3, the processing depicted in FIG. 4 may be performed by an information system and/or discovery platform.

At step 405, multi-omics data is collected from one or more data repositories. In some instances, the data and information relate to any source of multi-omics data such as private and public biological and/or in silico studies that encompass in vivo, in vitro, and computationally simulated experiments, respectively, that have generated the omics samples.

At step 410, the data collected from the data repositories is input into a discovery platform database where processing including: bioinformatic steps and data encoding and transformation are conducted in order to control the organization and structure of the multi-omics data.

At step 415, the various types of associated data (e.g., the normalized and reduced dimensional vectors and/or matrixes) are input into a discovery platform (A) comprising a cluster and regression analysis model, one or more machine learning models, and a multivariate dimensionality-reduction model.

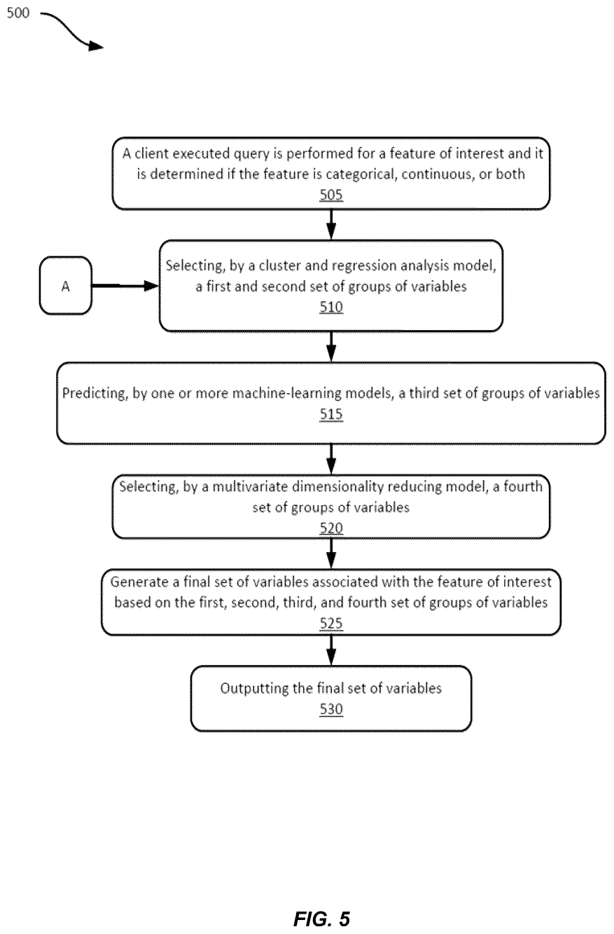

FIG. 5 is a flowchart illustrating a process 500 to predict/discover associated groups of variables corresponding to a feature of interest according to various embodiments. The processing depicted in FIG. 5 may be implemented in software (e.g., code, instructions, or programing) executed by one or more processing units (e.g., processors or cores) of the respective systems, hardware, or combinations thereof. The software may be stored on a non-transitory storage medium (e.g., on a memory device). The method presented in FIG. 5 and described below is intended to be illustrative and non-limiting. Although FIG. 5 depicts the various processing steps occurring in a particular sequence or order, this is not intended to be limiting. In certain alternative embodiments, the steps may be performed in a different order, or some steps may also be performed in parallel. In certain embodiments, such as in the embodiments depicted in FIGS. 1-3, the processing depicted in FIG. 5 may be performed by an information system and/or discovery platform.

At step 505, a query is received for a discovery platform configured to generate a final set of variables as an answer to the query. In some instances, a user (e.g., a customer) will have access to contents of the database(s) of the discovery platform via a user interface (restriction may occur) and will be able to navigate through the data. From there, the user can collect data as they see fit, formulate a query based on such data, and run the query through the workflow of analysis. In some instances, the user interface is user friendly and provides the user advice or recommendations on how to properly select or analyze the data. In certain instances, the discovery or query service could be provided as a cloud service (e.g., as a Software as a service (SaaS)). In other instances, a user can describe its request (e.g., problem to be solved) via the user interface to an expert associate with the discovery platform, and the expert can collect data as they see fit for completing the user's request, formulate a query based on such data, and run the query through the workflow of analysis.

The query comprises key terms and at least one feature of interest. Query terms (keywords) are the words contained in a user query. In some instances, the at least one feature of interest is a variable such as at least one categorical variable, at least one continuous variable, or a mixture of both. The query could target several features of interest at a same time. The features could then be a mix of categorical and continuous variables. This would mean that different paths in the workflow are executed to answer the query but, in reality, as long as the information is provided everything will be analyzed and all combinations of relations between variables will be outputted.

The discovery platform comprises a database, a data processing pipeline, and multiple analytical pipelines. The database comprises sets of multi-omics data. The sets of multi-omics data are generated by collecting raw data from one or more data repositories and processing the raw data using a data processing pipeline (see, e.g., process 400). The raw data relates to any source of multi-omics data such as private and public biological and/or in silico studies that encompass in vivo, in vitro, and computationally simulated experiments, respectively, that have generated the omics samples. The data processing pipeline comprises bioinformatic tools and a data encoding and transformation system. Each of the multiple analytical pipelines comprises cluster and regression analysis algorithms or models, one or more machine-learning models, and a multivariate dimensionality-reduction model.

At step 510, the query is executed on the database to retrieve variables discovered to be linked across the sets of multi-omics data that answer the query based on the key terms and the at least one feature of interest. Executing the query on the database comprises determining whether the variables discovered to be linked across the sets of multi-omics data answer the query based on the key terms and the at least one feature of interest. Determining whether the variables from the sets of multi-omics data answer the query comprises selecting those omics data with relevant biological information towards the query wherein all the biologically relevant data stored in the database is input for the discovery platform. In some instances, the variables from the sets of multi-omics data are encoded into high dimensional vectors and/or matrices and the high dimensional vectors and/or matrices are transformed, using data transformations, to generate normalized and reduced dimensional vectors and/or matrices.

At step 515, at least one of the multiple analytical pipelines is selected to be used for analyzing the variables based on the at least one feature of interest. Selecting the at least one of the multiple analytical pipelines to be used for analyzing the variables comprises choosing, either manually or through μl, the multiple analytical pipelines to run based on if the variable is at least one continuous, at least categorical, or a mixture of both. Optionally, all components of the analytical pipeline may be chosen. In some instances, selecting the at least one of the multiple analytical pipelines, comprises: determining whether each of the at least one feature of interest is at least one categorical variable, at least one continuous variable, or a mixture of both; and selecting the at least one of the multiple analytical pipelines to be used for analyzing the variables based on each of the at least one feature of interest being at least one categorical variable, at least one continuous variable, or a mixture of both.

At step 520, the variables are analyzed using the at least one of the multiple analytical pipelines to generate the final set of variables as the answer to the query. The analyzing comprises: (i) identifying, by the cluster and regression analysis algorithms or models, a first and second set of variables that are associated with the at least one feature of interest that take into account the variables and relationships between the variables; (ii) predicting, by the one or more machine-learning models, a third set of variables that are associated with the at least one feature of interest that take into account the variables and the relationships between the variables; and (iii) identifying, by the multivariate dimensionality-reduction model, a fourth set of variables that are associated with the at least one feature of interest that take into account the variables and the relationships between the variable. In some instances, the analysis further comprises inputting the normalized and reduced dimensional vectors and/or matrices into the at least one of the multiple analytical pipelines of the discovery platform.

In some instances, when the at least one feature of interest is the at least one categorical variable the one or more machine learning models comprise a Least Absolute Shrinkage and Selection Operator (LASSO) logit regression model, an elastic net penalized logit regression model where the penalization parameter is an optimized value, and a random forest or random decision forest classifier; and when the at least one feature of interest is the at least one continuous variable the one or more machine learning models comprise the LASSO regression model and the elastic net penalized regression model where the penalization parameter is an optimized value.

In certain instances, when the at least one feature of interest is the at least one categorical variable, the LASSO logit regression model predicts a first and second set of groups of the variables, the elastic net penalized logit regression model where the penalization parameter is an optimized value predicts a third set of groups of the variables, and the random forest or random decision forest classifier predicts a fourth set of groups of the variables, and the first prediction for a set of variables from the LASSO regression model, the second prediction for a set of variables from the elastic net penalized regression model where the penalization parameter is an optimized value, or the third prediction for a set of variables from the random forest or random decision forest classifier are graphed on a receiver operating characteristic curve, and the most robust prediction is selected as the third set of groups of variables.

In some instances, when the at least one feature of interest is the at least one continuous variable, the LASSO regression model predicts a first and second set of groups of the variables, the elastic net penalize regression model where the penalization parameter is an optimized value predicts a third set of groups of the variables, and the first prediction for a set of variables from the LASSO regression model, the second prediction for a set of variables from the elastic net penalized regression model where the penalization parameter is an optimized value, or the third prediction for a set of variables based on the mean squared error observed for each of the LASSO regression model are graphed on a receiver operating characteristic curve, and the most robust prediction is selected as the third set of groups of variables.

In some instances, when the at least one feature of interest is the at least one categorical variable the multivariate dimensionality-reduction model is a sparse partial least square—discriminant analysis model; and when the at least one feature of interest is the at least one continuous variable the multivariate dimensionality-reduction model is a sparse partial least square regression model.

At step 525, a final set of variables are generated that are correlated with the at least one feature of interest by cross-referencing at least the first set of variables, the second set of variables, the third set of variables, and the fourth set of variables. Cross-referencing at least the first set of variables, the second set of variables, the third set of variables, and the fourth set of variables to generate the final set of variables, comprises: choosing variables that convey the same biological information determined by core clustering and the use of biological information from previously known biological pathways.

At step 530, the final set of variables are output as the answer to the query. For example, the final set of variables may be communicated, transmitted, or displayed to a client as being correlated with the feature of interest through tables (e.g., OTUs, genes, molecules, and the like) that comprise information including correlations, p-values, ranking, and the like and through graphs connecting or quantifying the elements of the list/table (e.g., networks, trees, plots, etc.). Additionally or alternatively, the final set of variables may be stored in one or more data repositories for later retrieval and use (e.g., use in subsequent analysis by the discovery platform). In some instances, outputting the final set of variables comprises rendering the final set of variables in a graphical user interface as an answer to the query wherein the graphical user interface helps to interpret the data and display a selected set of final variables as the product based on the at least one feature of interest. In some instances, the final set of variables are used to manufacture a product wherein the final set of variables comprise a list of variables that when combined have a more significant biological impact compared to when they are alone; wherein the product comprises of variables that optimally perform better together. In some instances, the computer-implemented method provides a foundation for manufacturing the product, where the foundation comprises instructions on how to assemble the product in a correct order and proportions based on various components associated with the refined variables.

### VI. Additional Considerations

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps, and means described above can be done in various ways. For example, these techniques, blocks, steps, and means can be implemented in hardware, software, or a combination thereof. For a hardware implementation, the processing units can be implemented within one or more algorithm specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but there could be additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium”, “storage” or “memory” can represent one or more memories for storing data, including read only memory (ROM), random access memory (RAM), magnetic RAM, core memory, magnetic disk storage mediums, optical storage mediums, flash memory devices and/or other machine-readable mediums for storing information. The term “machine-readable medium” includes but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

What is claimed is:

1. A computer-implemented method comprising:
receiving a query for a discovery platform configured to generate a final set of variables as an answer to the query, wherein:
the query comprises key terms and at least one feature of interest,
the discovery platform comprises a database and multiple analytical pipelines,
the database comprises sets of processed multi-omics data, and
each of the multiple analytical pipelines comprises cluster and regression analysis algorithms or models, one or more machine learning models, and a multivariate dimensionality-reduction model;
executing the query on the database to retrieve variables discovered to be linked across the sets of multi-omics data that answer the query based on the key terms and the at least one feature of interest;
selecting at least one of the multiple analytical pipelines to be used for analyzing the variables based on the at least one feature of interest;
analyzing the variables using the at least one of the multiple analytical pipelines to generate the final set of variables as the answer to the query, wherein the analyzing comprises:
identifying, by the cluster and regression analysis algorithms or models, a first and second set of variables that are associated with the at least one feature of interest that take into account the variables and relationships between the variables;
predicting, by the one or more machine learning models, a third set of variables that are associated with the at least one feature of interest that take into account the variables and the relationships between the variables; and
identifying, by the multivariate dimensionality-reduction model, a fourth set of variables that are associated with the at least one feature of interest that take into account the variables and the relationships between the variable;
generating a final set of variables that are correlated with the at least one feature of interest by cross-referencing at least the first set of variables, the second set of variables, the third set of variables, and the fourth set of variables; and
outputting the final set of variables as the answer to the query.

2. The computer-implemented method of claim 1, further comprising:
encoding the variables from the sets of multi-omics data into high dimensional vectors and/or matrices; and
generating, by data transformations, normalized and reduced dimensional vectors and/or matrices based on the high dimensional vectors and/or matrices,
wherein the analyzing further comprises inputting the normalized and reduced dimensional vectors and/or matrixes into the at least one of the multiple analytical pipelines of the discovery platform.

3. The computer-implemented method of claim 1, wherein selecting the at least one of the multiple analytical pipelines, comprises:
determining whether each of the at least one feature of interest is at least one categorical variable, at least one continuous variable, or a mixture of both; and
selecting the at least one of the multiple analytical pipelines to be used for analyzing the variables based on each of the at least one feature of interest being at least one categorical variable, at least one continuous variable, or a mixture of both.

4. The computer-implemented method of claim 3, wherein when the at least one feature of interest is the at least one categorical variable the one or more machine learning models comprise a Least Absolute Shrinkage and Selection Operator (LASSO) logit regression model, an elastic net penalized logit regression model where the penalization parameter is an optimized value, and a random forest or random decision forest classifier; and when the at least one feature of interest is the at least one continuous variable the one or more machine learning models comprise the LASSO regression model and the elastic net penalized regression model where the penalization parameter is an optimized value.

5. The computer-implemented method of claim 4, wherein when the at least one feature of interest is the at least one categorical variable, the LASSO logit regression model predicts a first and second set of groups of the variables, the elastic net penalized logit regression model where the penalization parameter is an optimized value predicts a third set of groups of the variables, and the random forest or random decision forest classifier predicts a fourth set of groups of the variables, and the first prediction for a set of variables from the LASSO regression model, the second prediction for a set of variables from the elastic net penalized regression model where the penalization parameter is an optimized value, or the third prediction for a set of variables from the random forest or random decision forest classifier are graphed on a receiver operating characteristic curve, and the most robust prediction is selected as the third set of groups of variables.

6. The computer-implemented method of claim 3, wherein when the at least one feature of interest is the at least one continuous variable, the LASSO regression model predicts a first and second set of groups of the variables, the elastic net penalize regression model where the penalization parameter is an optimized value predicts a third set of groups of the variables, and the first prediction for a set of variables from the LASSO regression model, the second prediction for a set of variables from the elastic net penalized regression model where the penalization parameter is an optimized value, or the third prediction for a set of variables based on the mean squared error observed for each of the LASSO regression model are graphed on a receiver operating characteristic curve, and the most robust prediction is selected as the third set of groups of variables.

7. The computer-implemented method of claim 3, wherein when the at least one feature of interest is the at least one categorical variable the multivariate dimensionality-reduction model is a sparse partial least square—discriminant analysis model; and when the at least one feature of interest is the at least one continuous variable the multivariate dimensionality-reduction model is a sparse partial least square regression model.

8. The computer-implemented method of claim 1, wherein:
the sets of multi-omics data are generated by collecting raw data from one or more data repositories and processing the raw data using a data processing pipeline;
the raw data relates to any source of multi-omics data including private and public biological and/or in silico studies that encompass in vivo, in vitro, and computationally simulated experiments, respectively, that have generated the omics samples; and
the data processing pipeline comprises bioinformatic tools and a data encoding and transformation system.

9. The computer-implemented method of claim 1, wherein:
executing the query on the database comprises determining whether the variables from the sets of multi-omics data answer the query based on the key terms and the at least one feature of interest; and
determining whether the variables from the sets of multi-omics data answer the query comprises selecting those omics data with relevant biological information pertaining to the query wherein all the biologically relevant data stored in the database is input for the discovery platform.

10. The computer-implemented method of claim 1, wherein selecting the at least one of the multiple analytical pipelines to be used for analyzing the variables comprises choosing, either manually or through artificial intelligence, the multiple analytical pipelines to run based on if the variable is at least one continuous, at least one categorical, or a mixture of both.

11. The computer-implemented method of claim 1, wherein cross-referencing at least the first set of variables, the second set of variables, the third set of variables, and the fourth set of variables to generate the final set of variables, comprises: choosing variables that convey the same biological information determined by core clustering and the use of biological information from previously known biological pathways.

12. The computer-implemented method of claim 1, wherein outputting the final set of variables comprises rendering the final set of variables in a graphical user interface as an answer to the query wherein the graphical user interface helps to interpret the data and display a selected set of final variables as the product based on the at least one feature of interest.

13. A system comprising:
one or more processors; and
one or more computer-readable media storing instructions which, when executed by the one or more processors, cause the system to perform operations comprising:
receiving a query for a discovery platform configured to generate a final set of variables as an answer to the query, wherein:
the query comprises key terms and at least one feature of interest,
the discovery platform comprises a database and multiple analytical pipelines,
the database comprises sets of processed multi-omics data, and
each of the multiple analytical pipelines comprises cluster and regression analysis algorithms or models, one or more machine learning models, and a multivariate dimensionality-reduction model;
executing the query on the database to retrieve variables discovered to be linked across the sets of multi-omics data that answer the query based on the key terms and the at least one feature of interest;
selecting at least one of the multiple analytical pipelines to be used for analyzing the variables based on the at least one feature of interest;
analyzing the variables using the at least one of the multiple analytical pipelines to generate the final set of variables as the answer to the query, wherein the analyzing comprises:
identifying, by the cluster and regression analysis algorithms or models, a first and second set of variables that are associated with the at least one feature of interest that take into account the variables and relationships between the variables;
predicting, by the one or more machine learning models, a third set of variables that are associated with the at least one feature of interest that take into account the variables and the relationships between the variables; and
identifying, by the multivariate dimensionality-reduction model, a fourth set of variables that are associated with the at least one feature of interest that take into account the variables and the relationships between the variable;
generating a final set of variables that are correlated with the at least one feature of interest by cross-referencing at least the first set of variables, the second set of variables, the third set of variables, and the fourth set of variables; and
outputting the final set of variables as the answer to the query.

14. The system of claim 13, wherein the operations further comprise:
encoding the variables from the sets of multi-omics data into high dimensional vectors and/or matrices; and
generating, by data transformations, normalized and reduced dimensional vectors and/or matrices based on the high dimensional vectors and/or matrices,
wherein the analyzing further comprises inputting the normalized and reduced dimensional vectors and/or matrixes into the at least one of the multiple analytical pipelines of the discovery platform.

15. The computer-implemented method of claim 13, wherein selecting the at least one of the multiple analytical pipelines, comprises:
determining whether each of the at least one feature of interest is at least one categorical variable, at least one continuous variable, or a mixture of both; and
selecting the at least one of the multiple analytical pipelines to be used for analyzing the variables based on each of the at least one feature of interest being at least one categorical variable, at least one continuous variable, or a mixture of both.

16. The computer-implemented method of claim 15, wherein when the at least one feature of interest is the at least one categorical variable the one or more machine learning models comprise a Least Absolute Shrinkage and Selection Operator (LASSO) logit regression model, an elastic net penalized logit regression model where the penalization parameter is an optimized value, and a random forest or random decision forest classifier; and when the at least one feature of interest is the at least one continuous variable the one or more machine learning models comprise the LASSO regression model and the elastic net penalized regression model where the penalization parameter is an optimized value.

17. One or more non-transitory computer-readable media storing instructions which, when executed by one or more processors, cause a system to perform operations comprising:
receiving a query for a discovery platform configured to generate a final set of variables as an answer to the query, wherein:
the query comprises key terms and at least one feature of interest,
the discovery platform comprises a database and multiple analytical pipelines,
the database comprises sets of processed multi-omics data, and
each of the multiple analytical pipelines comprises cluster and regression analysis algorithms or models, one or more machine learning models, and a multivariate dimensionality-reduction model;
executing the query on the database to retrieve variables discovered to be linked across the sets of multi-omics data that answer the query based on the key terms and the at least one feature of interest;
selecting at least one of the multiple analytical pipelines to be used for analyzing the variables based on the at least one feature of interest;
analyzing the variables using the at least one of the multiple analytical pipelines to generate the final set of variables as the answer to the query, wherein the analyzing comprises:
identifying, by the cluster and regression analysis algorithms or models, a first and second set of variables that are associated with the at least one feature of interest that take into account the variables and relationships between the variables;
predicting, by the one or more machine learning models, a third set of variables that are associated with the at least one feature of interest that take into account the variables and the relationships between the variables; and
identifying, by the multivariate dimensionality-reduction model, a fourth set of variables that are associated with the at least one feature of interest that take into account the variables and the relationships between the variable;
generating a final set of variables that are correlated with the at least one feature of interest by cross-referencing at least the first set of variables, the second set of variables, the third set of variables, and the fourth set of variables; and
outputting the final set of variables as the answer to the query.

18. The computer-implemented method of claim 17, wherein the operations further comprise:
encoding the variables from the sets of multi-omics data into high dimensional vectors and/or matrices; and
generating, by data transformations, normalized and reduced dimensional vectors and/or matrices based on the high dimensional vectors and/or matrices,
wherein the analyzing further comprises inputting the normalized and reduced dimensional vectors and/or matrixes into the at least one of the multiple analytical pipelines of the discovery platform.

19. The computer-implemented method of claim 17, wherein selecting the at least one of the multiple analytical pipelines, comprises:
determining whether each of the at least one feature of interest is at least one categorical variable, at least one continuous variable, or a mixture of both; and
selecting the at least one of the multiple analytical pipelines to be used for analyzing the variables based on each of the at least one feature of interest being at least one categorical variable, at least one continuous variable, or a mixture of both.

20. The computer-implemented method of claim 19, wherein when the at least one feature of interest is the at least one categorical variable the one or more machine learning models comprise a Least Absolute Shrinkage and Selection Operator (LASSO) logit regression model, an elastic net penalized logit regression model where the penalization parameter is an optimized value, and a random forest or random decision forest classifier; and when the at least one feature of interest is the at least one continuous variable the one or more machine learning models comprise the LASSO regression model and the elastic net penalized regression model where the penalization parameter is an optimized value.
